(19) United States
(12) Patent Application Publication
(10) Pub. No.: US 2020/0375881 A1
(43) Pub. Date: Dec. 3, 2020

(54) Skin care product made from camellia sinensis and method of making the same

(71) Applicant: Yijian Shi, Mountain view, CA (US)

(72) Inventor: Yijian Shi, Mountain view, CA (US)

(21) Appl. No.: 16/877,489

(22) Filed: May 19, 2020

Related U.S. Application Data
(60) Provisional application No. 62/853,168, filed on May 28, 2019.

(51) Int. Cl. A61K 8/9789; A61K 8/02; A61Q 19/00

(52) U.S. Cl. CPC A61K 8/9789; A61K 8/0212; A61K 2800/10; A61Q 19/00; A61K 8/0216

(57) ABSTRACT

The subject invention provides a skin care product made from camellia sinensis, which is nontoxic and non-irritating to skin. More specifically, the present invention involves a skin care product containing an alcohol, acetic acid, and extracts of camellia sinensis. This product is effective in treating a number of skin conditions, including but not limited to seborrheic dermatitis, hair loss, athlete's foot, ichthyosis, jock itch, sweat spots, fungal nails, and burns. When applied to burns, it relieves pain and helps to subside blisters; when applied to sunburns, it eases skin itchiness and prevents peeling skin; when applied to the scalp, it helps to stop hair loss and regrows hair. Long-term use of this product will keep skin healthy and will not lead to any drug resistance or harmful side effects.

### CROSS-REFERENCE TO RELATED APPLICATION

This application claims priority under 35 U.S.C. §119(e) to U.S. Provisional Patent Application Ser. No. 62/853,168, filed May 28, 2019. The disclosure of the aforementioned application is herein incorporated by reference in its entirety.

### TECHNICAL FIELD

The present invention involves a skin care product made from camellia sinensis, which is used to treat a number of skin conditions. More specifically, the present invention involves a skin care product containing an alcohol, acetic acid, and extracts of camellia sinensis, which is effective in treating seborrheic dermatitis and a number of other common skin conditions including hair loss, athlete's foot, ichthyosis, jock itch, sweat spots, fungal nails, and bums.

### BACKGROUND OF THE INVENTION

Skin is the largest organ of human body, which protects the human body from microbes and harmful external influences. Once a pathological change or a wound is developed in the skin, bacteria and viruses may enter the human body via the lesion or the wound, which may result in serious health conditions. Therefore, healthy skin is very important to human health.

Seborrheic dermatitis is one of the most common skin diseases, which is a chronic inflammatory skin disorder characterized in immunocompetent adult patients by periods of exacerbation and remission. The reported prevalence of seborrheic dermatitis in the overall adult population is approximately 5 percent. The disorder can affect any ethnicity, and it appears to affect men more than women.

Another common skin condition is hair loss. Although historically, hair loss have been observed primarily in people of 50 years of age and older, recent studies have shown that the average age of patients is becoming younger and younger in recent years. According to a 2014 survey by the International Society of Hair Restoration Surgery (available at http://www.statisticbrain.com/ hair-loss-statistics), approximately 35 million men and 21 million women in the United States suffer hair loss, in which approximately 40 percent of men by the age of 35 are suffering from visible hair loss; according to a survey recently conducted by the Health Promotion and Education Association of China (available at https://wenku.baidu.com/view/cfffef21326c1eb91a37f111f18583d049640fbe.html), the majority of patients suffering hair loss fall in between the age of 20-40 years, and there is approximately a quarter of the Chinese adult males, equivalent to approximately 130 million, suffering hair loss.

Although the course of adult seborrheic dermatitis and hair loss in affected individuals is variable and its etiology is not definitely known, there are three principal factors that appear to play a role: sebaceous gland secretion, alteration in colonization and metabolism of cutaneous microflora, and individual susceptibility and host response.

For example, i) parasitic mites, fungus, bacteria, or virus in the scalp may results in inflammation of the scalp and the hair follicles, which results in hair loss; ii) intensive mental stress may result in autonomic or central nervous disorder, which results in seborrheic dermatitis and hair loss; iii) endocrine disorders may result in excessive secretion of androgens or sebum, resulting in excessive oleic acid, linoleic acid, squalene, and dihydrotestosterone in the scalp, which poison the hair follicles, resulting in hair thinning and hair loss.

The main goals of therapy for seborrheic dermatitis are to clear the visible signs of the disease and to ameliorate the associated symptoms, especially pruritus. As a result, the therapy usually involves antiseptic and antipruritic treatments.

Commonly used medications for treating seborrheic dermatitis include antifungal agents such as ketoconazole, ciclopirox, miconazole, terbinafine, bifonazole, antibiotics such as metronidazole, hormones such as hydrocortisone, and anti-infective agents such as selenium sulfide. Although these agents may be effective at ameliorating certain symptoms, they may cause a number of side effects at the application site, such as allergic inflammation of the skin, scalp and skin irritation, burning sensation, itching, scaling, skin thinning, etc. In some cases, drug resistance and hair loss may develop.

More information regarding to the treatment and medication for seborrheic dermatitis may be found in: i) James Q. Del Rosso, “Adult Seborrheic Dermatitis: A Status Report on Practical Topical Management”, J Clin Aesthet Dermatol., 2011 May, 4(5): 32-38; ii) Habif TP, et al. “Seborrheic dermatitis” in Dermatology DDx Deck, China, Mosby Elsevier, 2006, p. 40; iii) Plewig G and Jansen T. “Seborrheic Dermatitis” in Wolff K, et al., Fitzpatrick's Dermatology in General Medicine, 7th ed. United States of America, McGraw Hill Medical, 2008. p. 219-25.

Accordingly, there is a need for treatments of hair loss and seborrheic dermatitis that can effectively ameliorate the symptoms without causing harmful side effects. The present invention provides a skin care product made from camellia sinensis, which is nontoxic and non-irritating to skin. This product is effective in treating a number of skin conditions, including but not limited to seborrheic dermatitis and hair loss. In addition, long-term use of this product will effectively help to regrow hairs without developing any drug resistance or harmful side effects.

The skin care product described in the present invention is multifunctional: it is anti-inflammatory, antiseptic, antipruritic, and analgesic; it promotes self-healing of skin tissue and is effective in treating a number of skin conditions, including but not limited to seborrheic dermatitis, hair loss, athlete's foot, ichthyosis, jock itch, sweat spots, and fungal nails. When applied to scalp, it improves scalp health, stops hair loss, and regrows hair.

The skin care product described in the present invention is also effective in treating mild burns. When applied to burns, it relieves pain and subsides blisters; when applied to sunburns, it eases skin itchiness and prevents peeling skin.

### SUMMARY OF THE INVENTION

Aspects of the invention include the formulation of the skin care product and the method for making the same. In one embodiment, the skin care product is an aqueous solution containing, in mass percentage, the following components: 0.01-5% extract of camellia sinensis, 0.1-10% acetic acid, 5-40% alcohol, 50-95% water. Preferably, the skin care product contains, in mass percentage, the following components: 1-5% extract of camellia sinensis, 1-3% acetic acid, 5-20% alcohol, 80-90% water. In certain embodiments, the skin care product may also contain one or more of the following optional components: extract of onion, onion juice, extract of lemon, and lemon juice.

In certain embodiments, the aforementioned skin care product is further formulated and made into the form of a skin cream.

In certain embodiments, the aforementioned skin care product is further formulated and made into the form of a facial mask.

In certain embodiments, the extract of camellia sinensis used in the aforementioned skin care product is an alcohol extract of camellia sinensis, in which the active ingredient of camellia sinensis is obtained via an extraction process using an alcohol as the solvent.

In certain embodiments, the extract of camellia sinensis used in the aforementioned skin care product is an acid extract of camellia sinensis, in which the active ingredient of camellia sinensis is obtained via an extraction process using an acid as the solvent.

In certain embodiment, the extract of camellia sinensis used in the aforementioned skin care product is a water extract of camellia sinensis, in which the active ingredient of camellia sinensis is obtained via an extraction process using water as the solvent.

In certain embodiments, the extract of camellia sinensis used in the aforementioned skin care product is obtained via an extraction process using a mixed solvent, for instance a mixture of alcohol, acid, and water.

In certain embodiments, the extract of camellia sinensis used in the aforementioned skin care product is a mixture of the alcohol extract, acid extract, and water extract of camellia sinensis. Accordingly, the aforementioned skin care product contains, in mass percentage, the following components: 0.01-5% alcohol extract of camellia sinensis, 0.01-5% acid extract of camellia sinensis, 0.01-5% water extract of camellia sinensis, 0.1-10% acetic acid, 5-40% alcohol, 50-95% water. Preferably, the skin care product contains, in mass percentage, the following components: 1% alcohol extract of camellia sinensis, 0.5% acid extract of camellia sinensis, 1% water extract of camellia sinensis, 1-3% acetic acid, 5-20% alcohol, and 80-90% water. The skin care product may also contain one or more of the following optional components: an onion juice, an extract of onion, a lemon juice, and an extract of lemon. In certain embodiments, the skin care product is further formulated and made into the form of a skin cream. In certain embodiments, the skin care product is further formulated and made into the form of a facial mask.

In another embodiment, the present invention provides a method for producing the aforementioned skin care product, which involves the following steps: in mass ratio, 10 part of camellia sinensis, 1-10 acetic acid, 50-200 parts of alcohol, and 200-600 parts of purified water is mixed and heated in a reactor to reflux for 2 hours; then the mixture is cooled to 70-80° C., and the solid residue is removed by filtration to yield the aforementioned skin care product. In certain embodiments, one or more of the optional components (onion juice or extract of onion, lemon juice or extract of lemon) is added into the final product after the filtration step. In certain embodiments, the skin care product is further formulated and made into the form of a skin cream or into the form of a facial mask.

In another embodiment, the present invention provides another method for producing the aforementioned skin care product, which involves the following steps: i) produce the necessary extract of camellia sinensis; ii) optionally, produce one or more of the following components—lemon juice, onion juice, extract of onion, and extract of lemon; iii) mix the extract of camellia sinensis with acetic acid, alcohol, water, and the optional component, if there is any in the selected formulation, in a preferred ratio.

Therefore, in another embodiment, the present invention provides a method for producing the alcohol extract of camellia sinensis required by the formulation of the aforementioned skin care product, which involves the following steps: i) in mass ratio, one part of camellia sinensis and 10-30 parts of alcohol is mixed in a sealed container and heated to 70-80° C. for 1-2 hours, or alternatively the mixture is refluxed under atmosphere for 1-2 hours; ii) the solid residue is removed by filtration to yield a solution of the alcohol extract of camellia sinensis; iii) the solution (or the filtrate) is subjected to reduced pressure distillation to have the solvent removed either completely to yield the pure alcohol extract of camellia sinensis, or partially to yield a more concentrated solution that is suitable for later applications. In certain embodiments, the extraction process is performed at a lower temperature with extended time to reduce energy consumption. For example, instead of heating the mixture continuously for 1-2 hours, the first step may be modified as follows: either a) keeping the mixture at room temperature for several days, or b) heating the mixture to 60-70° C. and subsequently keeping it in a heat insulated container for 12 hours.

In another embodiment, the present invention provides a method for producing the acid extract of camellia sinensis required by the formulation of the aforementioned skin care product, which involves the following steps: i) in mass ratio, one part of camellia sinensis and 10-30 parts of an acid solution is mixed in a sealed container and heated to 80-105° C. for 1-2 hours, or alternatively the mixture is refluxed under atmosphere for 1-2 hours; ii) the solid residue is removed by filtration to yield a solution of the acid extract of camellia sinensis; iii) the solution (or the filtrate) is subjected to reduced pressure distillation to have the solvent removed either completely to yield the pure acid extract of camellia sinensis, or partially to yield a more concentrated solution that is suitable for later applications. In certain embodiments, the extraction process is performed at a lower temperature with extended time to reduce energy consumption. For example, instead of heating the mixture continuously for 1-2 hours, the first step may be modified as: heating the mixture to 70-80° C. and subsequently keeping it in a heat insulated container for 12 hours.

In another embodiment, the present invention provides a method for producing the water extract of camellia sinensis required by the formulation of the aforementioned skin care product, which involves the following steps: i) in mass ratio, one part of camellia sinensis and 10-30 parts of water is mixed in a sealed container and heated to 80-100° C. for 1-2 hours, or alternatively the mixture is refluxed under atmosphere for 1-2 hours; ii) the solid residue is removed by filtration to yield a solution of the water extract of camellia sinensis; iii) the solution is subjected to reduced pressure distillation to have the solvent removed either completely to yield the pure water extract of camellia sinensis, or partially to yield a more concentrated solution that is suitable for later applications. In certain embodiments, the extraction process is performed at a lower temperature with extended time to reduce energy consumption. For example, instead of heating the mixture continuously for 1-2 hours, the first step may be modified as: heating the mixture to 70-80° C. and subsequently keeping it in a heat insulated container for 12 hours.

In another embodiment, the present invention provides a method for producing an extract of onion, which involves the following steps: i) in mass ratio, one part of onion is soaked in 1-3 parts of an alcohol in a sealed container at room temperature for several days; ii) the solid residue is removed by filtration to yield a solution of the extract of onion. Alternatively, in order to reduce the processing time, the method may be modified as follows: a) in mass ratio, one part of onion is pressed and ground, then mixed with 1-3 parts of an alcohol, and then filtered to yield a solution of the extract of onion; or b) in mass ratio, one part of onion and 1-3 parts of an alcohol is mixed, then sonicated for 30 minutes, and then filtered to yield a solution of the extract of onion.

In another embodiment, the present invention provides a method for producing the extract of lemon, which involves the following steps: i) in mass ratio, one part of lemon is soaked in 1-3 parts of an alcohol in a sealed container at room temperature for several days; ii) the solid residue is removed by filtration to yield a solution of the extract of lemon. Alternatively, in order to reduce the processing time, the method may be modified as follows: a) in mass ratio, one part of lemon is pressed and ground, then mixed with 1-3 parts of an alcohol, and then filtered to yield a solution of the extract of lemon; or b) in mass ratio, one part of lemon and 1-3 parts of an alcohol is mixed, then sonicated for 30 minutes, and then filtered to yield a solution of the extract of lemon.

In certain embodiments, the aforementioned skin care product is further formulated and made into the form of a skin cream. In certain embodiments, the skin care product is further formulated and made into the form of a facial mask.

In certain embodiments, the camellia sinensis used in producing the aforementioned skin care product is in the form of a tea.

Preferably, the camellia sinensis used in producing the aforementioned skin care product is a non-fermented or lightly fermented tea.

Preferably, the camellia sinensis used in producing the aforementioned skin care product is the Baicha (White tea),

Preferably, the camellia sinensis used in producing the aforementioned skin care product is the Maocha (Mao tea).

### BRIEF DESCRIPTION OF THE FIGURES

FIG. 1 compares the human facial skin before (left image) and after (right image) using this skin care product. The left image shows the original appearance of an unhealthy human facial skin (male, aged 55-60) that is full of deep wrinkles and dry flaky particles. Application of this skin care product removes all the wrinkles and most of the flaky particles (right image) within 1-2 minutes, and the skin recovers its smoothness and healthy appearance.

FIG. 2 shows the changes in human skin before (left image) and after (right image) applying this skin care product: more than half of the skin grooves in the left image have been eliminated within a few minutes after application of this skin care product (right image); for those remaining skin grooves, the length, depth, and width are significantly reduced if compared to those in the left image. As a result, skin grooves become less visible and the skin appears smoother after application of the skin care product. Both images were taken at the same spot of a human forearm (female, aged 40-45) under an optical microscope with 100× magnification.

FIG. 3 shows the results of treating scalp seborrheic dermatitis (male, aged 45-50) with this skin care product. The left image was taken before the treatment, in which patches of reddish greasy skin covered with flaky white scales were visible. The symptoms were eliminated after treatment for two weeks (right image).

FIG. 4 shows another example (female, aged 53) of treating scalp seborrheic dermatitis with this skin care product. The left image was taken before the treatment, in which the scalp was covered with flaky white scales. The symptoms were eliminated and the scalp recovered its normal and healthy appearance after using the skin care product for three weeks (right image).

FIG. 5 shows an example of hair regrowth with this skin care product. The patient (male, aged 35) has suffered severe hair loss for several years (left image). After applying this product over the scalp on a daily basis for 6 months, new hairs of 1-2 cm long with relatively high density appear in the area originally without hair (right image).

FIG. 6 shows another example of hair regrowth with this skin care product: it is a plot of hair density versus time for a patient (female, aged 55-60) who has been applying this skin care product to her scalp on a daily basis. The plot shows progressive increases of patient's hair density. After treating the scalp for approximately 13 months, patient's hair density increases from 108/cm² to 141/cm², which is a 30% gain in hair density.

FIG. 7 shows another example of hair regrowth with this skin care product: it is a plot of hair density versus time for a senior patient (female, aged 80-85) who had been applying this skin care product to her scalp on a daily basis. The plot shows progressive increase of patient's hair density. After treating the scalp for approximately 8 months, patient's hair density increases from 46/cm² to 73/cm², which is a 59% gain in hair density.

FIG. 8 shows an example of treating burn blisters with this skin care product. The left image is taken before the treatment, where burn blisters are clearly visible. After applying this skin care product to the burned area, the fluid is re-absorbed and blisters subside in approximately 2 hours (right image).

FIG. 9 shows another example of treating burns with this skin care product. The left image is taken before the treatment, where skin whitening (and hardening) caused by burning is shown; after applying this skin care product to the burned area, the skin recovers its normal appearance (color) and softness in a few hours (right image).

FIG. 10 shows an example of subsiding swelling with this skin care product. The patient (female, 50-55) had a surgery at the right ankle joint and the right leg/foot was cast for several weeks after the surgery. The left image was taken on the first day when the cast was removed, which showed significant swelling. Generally, it would take several weeks for the swelling to subside naturally. However, after applying this product to the right foot surface several times, the swelling subsides within 12 hours (right image).

Before the present invention is further described, it is to be understood that this invention is not limited to particular embodiments described, and as such may of course vary. It is also to be understood that the terminology used herein is for the purpose of describing particular embodiments only, and is not intended to be limiting. Unless defined otherwise, all technical and scientific terms used herein have the same meaning as commonly understood by one skilled in the art to which this invention belongs.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Throughout this application, various publications, patents and published patent applications are cited. The disclosures of these publications, patents and published patent applications referenced in this application are hereby incorporated by reference in their entirety into the present disclosure. Citation herein by the Applicant of a publication, patent, or published patent application is not an admission by the Applicant of said publication, patent, or published patent application as prior art.

It must be noted that as used herein and in the appended claims, the singular forms “a”, “and”, and “the” include plural referents unless the context clearly dictates otherwise. Thus, for example, reference to “an extract of camellia sinensis” encompasses more than one extract of camellia sinensis, and also encompasses a mixture of more than one extract of camellia sinensis, and the like. It is further noted that the claims may be drafted to exclude any optional element. As such, this statement is intended to serve as antecedent basis for use of such exclusive terminology as “solely”, “only” and the like, in connection with the recitation of claim elements, or the use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The term “camellia sinensis” used herein refers to the camellia sinensis plant and any product derived from the camellia sinensis plant such as a tea. Camellia sinensis plant is a species of evergreen shrubs or small trees in the flowering plant family Theaceae whose leaves and leaf buds are used to produce tea.

The term “tea” is used in its ordinary sense and refers to a product derived from the leaves, buds, and stems of the camellia sinensis plant. Tea is generally divided into different categories based on how it is processed. In its most general form, tea processing involves withering, bruising, oxidizing, fixing and drying. When the manner and degree of each step varies, different types of tea result.

The term “extraction” used herein refers to a process to separate a desired substance when it is mixed with others. In an extraction process, the mixture or the starting material is brought into contact with a solvent in which the substance of interest is soluble, but the other substances present are insoluble. More specifically, the term “extraction” used throughout this application refers to a process of soaking the starting material, such as a tea or camellia sinensis, in a solvent at a certain temperature for a certain period of time, followed by filtration to remove the unwanted solid residues, where the desired substance is dissolved in the liquid (filtrate) obtained after the filtration step.

The term “filtrate” is used herein to refer the liquid obtained after a filtration process.

The term “extract” is used herein to refer the product of an extraction process. More specifically, the term “extract” used throughout this application refers to the filtrate and/or the substance contained in the filtrate. It should be noted that when the term “extract” refers to the filtrate, it is sometimes expressed as “a solution of the extract”, “the solution form of the extract”, or the like, and in all cases these terminologies used herein have the same meaning. It should also be noted that when the term “extract” refers to the substance contained in the filtrate, it is sometimes expressed as “pure form of the extract”, “pure extract”, or the like, and in these cases the word “pure” means that the majority of the solvent in the filtrate has been removed, and does not necessarily mean that the extract contains only a single substance.

In describing an extract obtained from a specific solvent and in distinguishing extracts obtained from different solvents, a combination of the name of solvent and the word “extract” is used herein. For instance, “alcohol extract of camellia sinensis” indicates that the extract of camellia sinensis is obtained by using an alcohol as the solvent for the extraction process; “water extract of camellia sinensis” indicates that the extract of camellia sinensis is obtained by using water as the solvent for the extraction process; “acid extract of camellia sinensis” indicates that the extract of camellia sinensis is obtained by using an acid as the solvent for the extraction process.

The term “alcohol” used herein refers to any of a class of organic compounds characterized by one or more hydroxyl (—OH) groups attached to a carbon atom of an alkyl group (hydrocarbon chain) and the aqueous solution containing such compounds. Thus, in describing the solvent used for the extraction process, the term “alcohol” refers to any of such organic compounds as well as a mixture of water and such compounds. Some examples for an alcohol used in the extraction process include but not limited to 100% methanol, 95% ethanol (containing 5% water), 75% ethanol (containing 25% water), 35% ethanol (containing 65% water), 70% 2-propanol (containing 30% water), 35% 2-propanol (containing 65% water), 35% 1-propanol (containing 65% water), and a wine.

The term “acid” used herein refers to a compound that releases hydrogen ion (H⁺) in water and a solution of such compound in water. Thus, in describing a solvent used for the extraction process, the term “acid” refers to any of such compounds as well as a mixture of water and such compounds. Some examples for an acid include but not limited to hydrochloric acid (a water solution of hydrogen chloride), dilute sulfuric acid (a water solution of sulfur trioxide), 10% phosphoric acid (containing 90% water), 95% acetic acid (containing 5% water), 30% acetic acid (containing 70% water), 15% acetic acid (containing 85% water), 5% acetic acid (containing 95% water) or vinegar.

“Optional” or “optionally” means that the subsequently described event or circumstance may or may not occur, and that the description includes instances where said event or circumstance occurs and instances where it does not. For example, the skin care product containing an “optional extract of onion” means that the extract of onion may or may not be present and that the description includes either state.

Other definitions of basic organic chemical terms may be found in organic chemistry textbooks, for example, in Marc Loudon and James Parise, Organic Chemistry (Roberts and Company Publishers Pub, 6^{th} ed. 2015). Reference may be also made to Ruth Winter, A Consumer's Dictionary of Cosmetic Ingredients (Harmony, 7^{th} ed. 2009).

### DETAILED DESCRIPTION

Aspects of the invention include the formulation of the skin care product and the method for making the same. In one embodiment, the skin care product is an aqueous solution containing, in mass percentage, the following components: 0.01-5% extract of camellia sinensis, 0.1-10% acetic acid, 5-40% alcohol, 50-95% water. Preferably, the skin care product contains, in mass percentage, the following components: 1-5% extract of camellia sinensis, 1-3% acetic acid, 5-20% alcohol, 80-90% water. In certain embodiments, the skin care product may also contain one or more of the following optional components: 0.01-5% extract of onion, 5-20% onion juice, 0.01-5% extract of lemon, and 5-20% lemon juice.

In certain embodiments, the aforementioned skin care product is further formulated and made into the form of a skin cream.

In certain embodiments, the aforementioned skin care product is further formulated and made into the form of a facial mask.

In certain embodiments, the extract of camellia sinensis used in the aforementioned skin care product is an alcohol extract of camellia sinensis, in which the active ingredient of camellia sinensis is obtained via an extraction process using an alcohol as the solvent.

In certain embodiments, the extract of camellia sinensis used in the aforementioned skin care product is an acid extract of camellia sinensis, in which the active ingredient of camellia sinensis is obtained via an extraction process using an acid as the solvent.

In certain embodiments, the extract of camellia sinensis used in the aforementioned skin care product is a water extract of camellia sinensis, in which the active ingredient of camellia sinensis is obtained via an extraction process using water as the solvent.

In certain embodiments, the extract of camellia sinensis used in the aforementioned skin care product is obtained via an extraction process using a mixed solvent, for instance a mixture of alcohol, acid, and water.

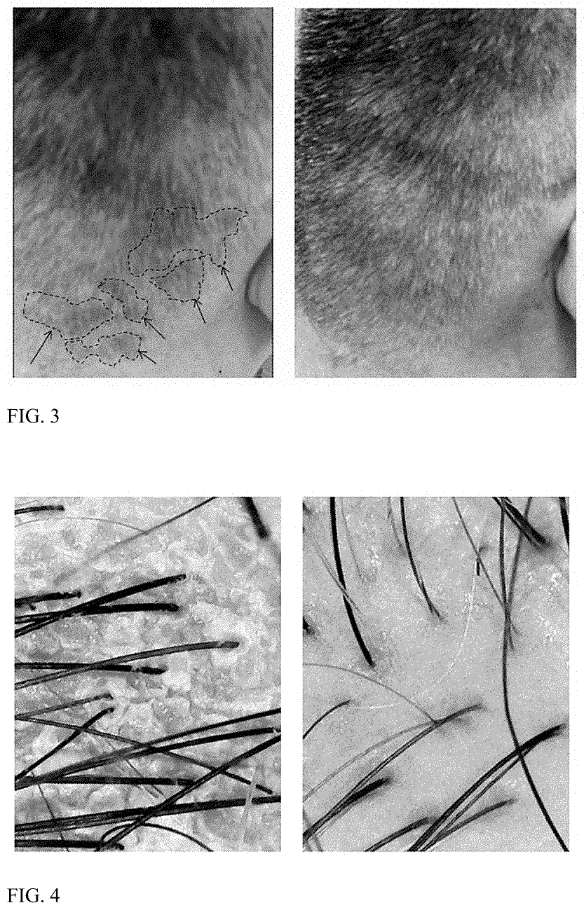

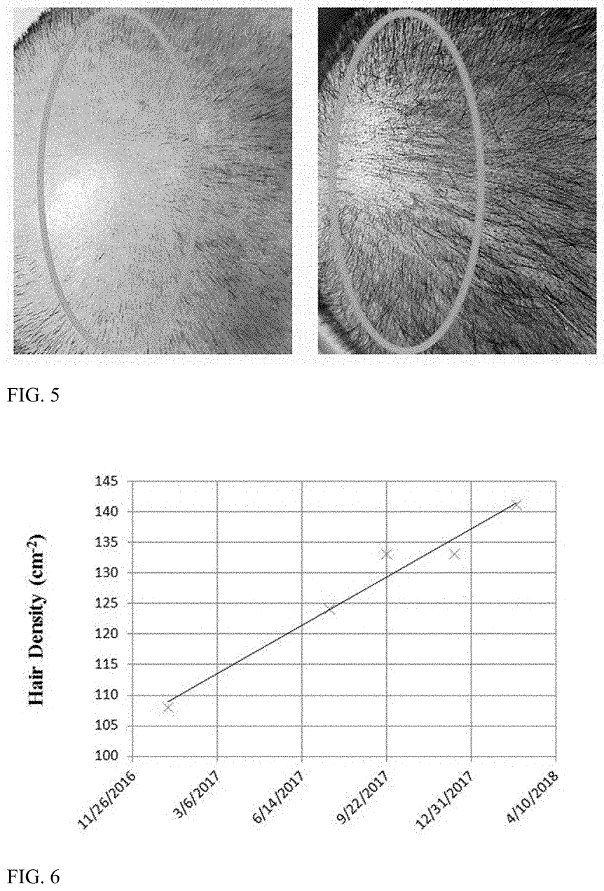

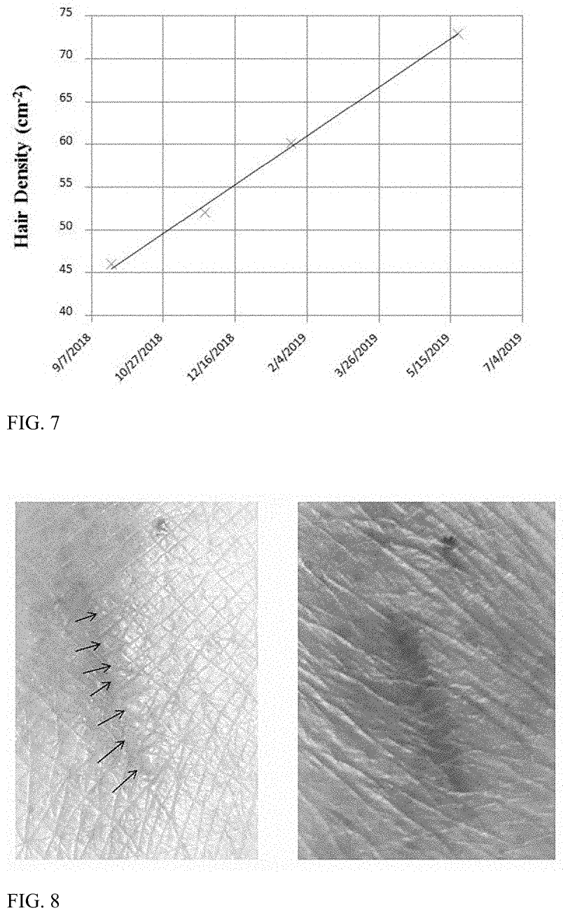

In certain embodiments, the extract of camellia sinensis used in the aforementioned skin care product is a mixture of the alcohol extract, acid extract, and water extract of camellia sinensis. For instance, in order to strengthen its efficacy in treating scalp seborrheic dermatitis, the aforementioned skin care product is accordingly formulated to contain, in mass percentage, the following components: 0.01-5% alcohol extract of camellia sinensis, 0.01-5% acid extract of camellia sinensis, 0.01-5% water extract of camellia sinensis, 0.1-10% acetic acid, 5-40% alcohol, 50-95% water. Preferably, the skin care product contains, in mass percentage, the following components: 1% alcohol extract of camellia sinensis, 0.5% acid extract of camellia sinensis, 1% water extract of camellia sinensis, 1-3% acetic acid, 5-20% alcohol, 80-90% water. The resulting skin care product effectively ameliorates the symptoms of seborrheic dermatitis (FIGS.3-4), improves skin health, and stops hair loss. As a result, for those who suffer severe hair loss, applying this product on scalp regularly on a daily basis for a certain period of time (generally, 6 months or more is required) may significantly increase their hair density (FIGS. 5-7).

In certain embodiments, the aforementioned skin care product contains the following optional component: an extract of onion. Accordingly, the skin care product contains, in mass percentage, the following components: 0.01-5% extract of camellia sinensis, 0.01-5% extract of onion, 0.1-10% acetic acid, 5-40% alcohol, 50-95% water.

In certain embodiments, the aforementioned skin care product contains the following optional component: an onion juice. Accordingly, the skin care product contains, in mass percentage, the following components: 0.01-5% extract of camellia sinensis, 5-20% onion juice, 0.1-10% acetic acid, 5-40% alcohol, 50-95% water.

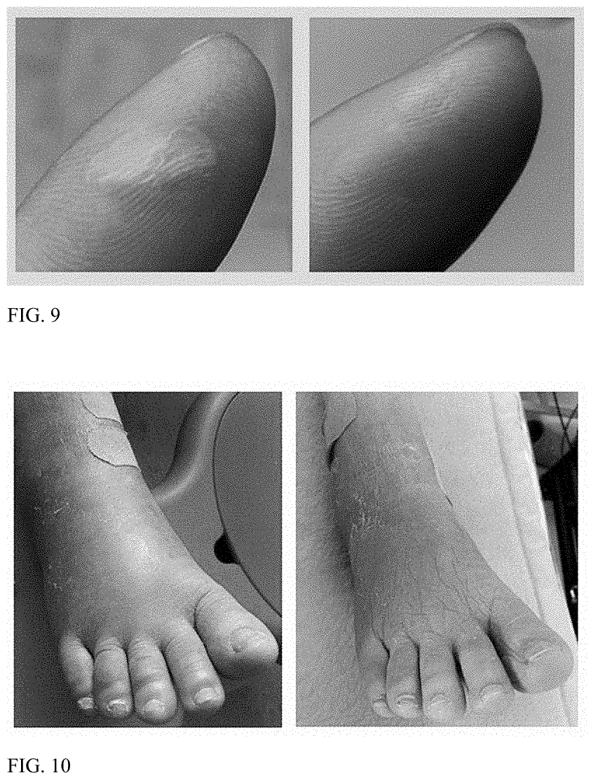

The aforementioned skin care product containing the optional onion juice or extract of onion effectively subsides swelling (FIG. 10) and blisters (FIG. 8), eases pain, and softens hardened skins caused by burns (FIG. 9); when applied to sunburns, it eases skin itchiness and prevents peeling skin.

In certain embodiments, the aforementioned skin care product contains the following optional component: an extract of lemon. Accordingly, the skin care product contains, in mass percentage, the following components: 0.01-5% extract of camellia sinensis, 0.01-5% extract of lemon, 0.1-10% acetic acid, 5-40% alcohol, 50-95% water;

In certain embodiments, the aforementioned skin care product contains the following optional component: a lemon juice. Accordingly, the skin care product contains, in mass percentage, the following components: 0.01-5% extract of camellia sinensis, 5-20%lemon juice, 0.1-10% acetic acid, 5-40% alcohol, 50-95% water.

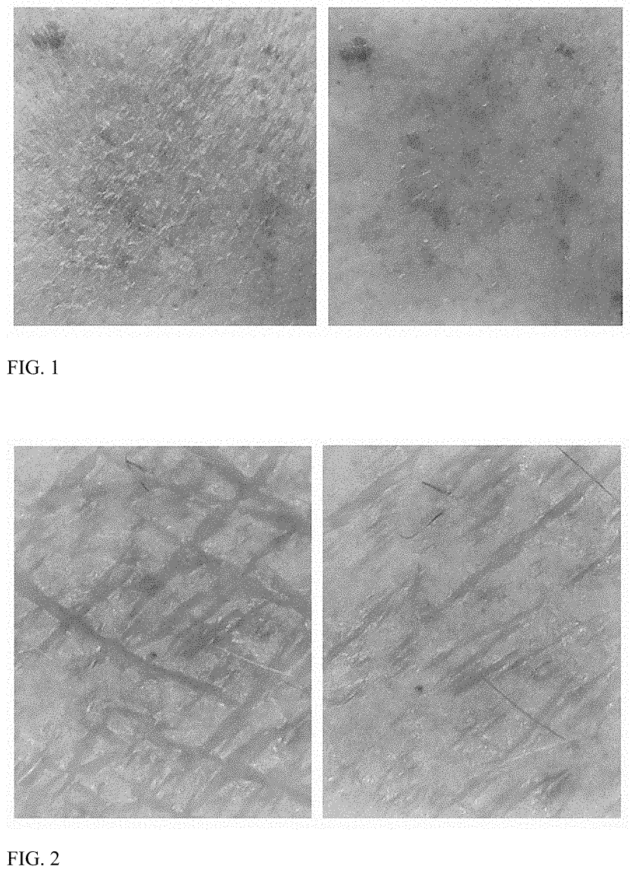

The skin care product containing the optional lemon juice or extract of lemon is especially effective in improving skin texture. An example is shown in FIG. 1, in which an unhealthy human facial skin that is full of deep wrinkles and dry flaky particles is shown (left image). After application of this skin care product, the skin recovers its smoothness and healthy appearance almost instantly (right image). FIG. 2 shows another example of improving the skin texture: before application of the skin care product, the skin has more skin grooves and the skin grooves are longer, wider, deeper, and more visible (left image). After the skin care product is applied, more than half of the skin grooves have become invisible or barely visible, and the size (length, width, and depth) of the remaining skin grooves is significantly reduced.

In certain embodiments, the aforementioned skin care product is formulated and made into the form of a skin cream.

In certain embodiments, the aforementioned skin care product is formulated and made into the form of a facial mask.

In another embodiment, the present invention provides a method for producing the aforementioned skin care product, which involves the following steps: in mass ratio, 10 part of camellia sinensis, 1-10 acetic acid, 50-200 parts of alcohol, and 200-600 parts of purified water is mixed and heated in a reactor to reflux for 2 hours; then the mixture is cooled to 70-80° C., and the solid residue is removed by filtration to yield the aforementioned skin care product. In certain embodiments, the skin care product is further formulated to contain one or more of the following optional components: 5-20% onion juice or 0.01-5% extract of onion, 5-20% lemon juice or 0.01-5% extract of lemon.

In another embodiment, the present invention provides another method for producing the aforementioned skin care product, which involves the following steps:
1) Produce the necessary extract of camellia sinensis;
2) Optionally, produce one or more of the following optional components if it is required by the specific formulation: a lemon juice or an extract of lemon, an onion juice or an extract of onion;
3) Mix the extract of camellia sinensis with acetic acid, alcohol, water, and the optional component in a predetermined ratio.

Thus, in another embodiment, the present invention provides a method for producing the alcohol extract of camellia sinensis required by the formulation of the aforementioned skin care product, which involves the following steps: i) in mass ratio, one part of camellia sinensis and 10-30 parts of alcohol is mixed in a sealed container and heated to 70-80° C. for 1-2 hours, or alternatively the mixture is refluxed under atmosphere for 1-2 hours; ii) the solid residue is removed by filtration to yield a solution of the alcohol extract of camellia sinensis; iii) the solution is subjected to reduced pressure distillation to have the solvent removed either completely to yield the pure alcohol extract of camellia sinensis, or partially to yield a more concentrated solution that is suitable for later applications. In certain embodiments, the extraction process is performed at a lower temperature with extended time to reduce energy consumption. For example, instead of heating the mixture continuously for 1-2 hours, the first step may be modified as follows: either a) keeping the mixture at room temperature for several days, or b) heating the mixture to 60-70° C. and subsequently keeping it in a heat insulated container for 12 hours.

In another embodiment, the present invention provides a method for producing the acid extract of camellia sinensis required by the formulation of the aforementioned skin care product, which involves the following steps: i) in mass ratio, one part of camellia sinensis and 10-30 parts of an aqueous acid solution is mixed in a sealed container and heated to 80-105 C for 1-2 hours, or alternatively the mixture is refluxed under atmosphere for 1-2 hours; ii) the solid residue is removed by filtration to yield a solution of the acid extract of camellia sinensis; iii) the solution is subjected to reduced pressure distillation to have the solvent removed either completely to yield the pure acid extract of camellia sinensis, or partially to yield a more concentrated solution that is suitable for later applications. In certain embodiments, the extraction process is performed at a lower temperature with extended time to reduce energy consumption. For example, instead of heating the mixture continuously for 1-2 hours, the first step may be modified as: heating the mixture to 70-80° C. and subsequently keeping it in a heat insulated container for 12 hours.

In another embodiment, the present invention provides a method for producing the water extract of camellia sinensis required by the formulation of the aforementioned skin care product, which involves the following steps: i) in mass ratio, one part of camellia sinensis and 10-30 parts of water is mixed in a sealed container and heated to 80-100° C. for 1-2 hours, or alternatively the mixture is refluxed under atmosphere for 1-2 hours; ii) the solid residue is removed by filtration to yield a solution of the water extract of camellia sinensis; iii) the solution is subjected to reduced pressure distillation to have the solvent removed either completely to yield the pure water extract of camellia sinensis, or partially to yield a more concentrated solution that is suitable for later applications. In certain embodiments, the extraction process is performed at a lower temperature with extended time to reduce energy consumption. For example, instead of heating the mixture continuously for 1-2 hours, the first step may be modified as: heating the mixture to 70-80° C. and subsequently keeping it in a heat insulated container for 12 hours.

In another embodiment, the present invention provides a method for producing the extract of onion optionally required by the formulation of the aforementioned skin care product, which involves the following steps: i) in mass ratio, one part of onion is soaked in 1-3 parts of an alcohol in a sealed container at room temperature for several days; ii) the solid residue is removed by filtration to yield a solution of the extract of onion. Alternatively, in order to reduce the processing time, the method may be modified as follows: a) in mass ratio, one part of onion is pressed and ground, then mixed with 1-3 parts of an alcohol, then filtered to yield a solution of the extract of onion; or b) in mass ratio, one part of onion and 1-3 parts of an alcohol is mixed, then sonicated for 30 minutes, and then filtered to yield a solution of the extract of onion.

In another embodiment, the present invention provides a method for producing the extract of lemon optionally required by the formulation of the aforementioned skin care product, which involves the following steps: i) in mass ratio, one part of lemon is soaked in 1-3 parts of an alcohol in a sealed container at room temperature for several days; ii) the solid residue is removed by filtration to yield a solution of the extract of lemon. Alternatively, in order to reduce the processing time, the method may be modified as follows: a) in mass ratio, one part of lemon is pressed and ground, then mixed with 1-3 parts of an alcohol, then filtered to yield a solution of the extract of lemon; or b) in mass ratio, one part of lemon and 1-3 parts of an alcohol is mixed, then sonicated for 30 minutes, and then filtered to yield a solution of the extract of lemon.

In certain embodiments, the aforementioned skin care product is further formulated and made into the form of a skin cream.

In certain embodiments, the aforementioned skin care product is further formulated and made into the form of a facial mask.

In certain embodiments, the camellia sinensis used in producing the aforementioned skin care product is in the form of a tea.

Preferably, the camellia sinensis used in producing the aforementioned skin care product is a non-fermented or lightly fermented tea.

Preferably, the camellia sinensis used in producing the aforementioned skin care product is the Baicha (White tea),

Preferably, the camellia sinensis used in producing the aforementioned skin care product is the Maocha (Mao tea).

### EXAMPLES

### Example 1

An example of preparation of a skin care product containing, in mass percentage, the following components: 0.01-5% extract of camellia sinensis, 0.1-10% acetic acid, 5-40% alcohol, 50-95% water, involves the following steps:

(1) In mass ratio, one part of tea and 30 parts of 70% ethanol are added into a reactor and refluxed for 1.5 hours; the solid residue is subsequently removed by filtration; the solvent is then removed by reduced pressure distillation to yield the alcohol extract of camellia sinensis.

(2) In mass ratio, one part of tea and 30 parts of purified water are added into a reactor and refluxed for 1.5 hours; the solid residue is subsequently removed by filtration; the solvent is then removed by reduced pressure distillation to yield the water extract of camellia sinensis.

(3) In mass ratio, one part of tea and 30 parts of 10% acetic acid solution (containing 90% water) are added into a reactor and refluxed for 1.5 hours; the solid residue is subsequently removed by filtration; the solvent is then removed by reduced pressure distillation to yield the acid extract of camellia sinensis.

(4) 1.0 gram of the alcohol extract of camellia sinensis (product of Step 1) is dissolved in 30 mL of 95% ethanol; 1.0 gram of the water extract of camellia sinensis (product of Step 2) is dissolved in 170 mL of purified water; 0.5 gram of the acid extract of camellia sinensis (product of Step 3) is dissolved in 100 mL of vinegar (5% acetic acid solution).

(5) The three solutions obtained from Step 4 are mixed to give the aforementioned skin care product.

### Example 2

A second example of preparation of the aforementioned skin care product involves the following steps:

1) In mass ratio, 10 parts of tea, 5 parts acetic acid, 50 parts of ethanol, and 300 parts of purified water is added into a reactor;

2) The reactor is heated to reflux for 2 hours; then the mixture is cooled to 70-80° C. and filtered to remove the solid residue.

3) The skin care product is obtained after addition of 3 parts of acetic acid into the filtrate obtained from Step 2.

### Example 3

A third example of preparation of the aforementioned skin care product involves the following steps:

(1) In mass ratio, 5 parts of tea and 100 parts of 70% ethanol are added into a reactor and refluxed for 1.5 hours; the solid residue is subsequently removed by filtration to yield a solution of the alcohol extract of camellia sinensis.

(2) In mass ratio, 5 part of tea and 200 parts of vinegar (or 5% acetic acid solution) are added into a reactor and refluxed for 1.5 hours; the solid residue is subsequently removed by filtration to yield a solution of the acid extract of camellia sinensis.

(3) In mass ratio, 10 parts of tea and 200 parts of purified water are added into a reactor and refluxed for 1.5 hours; the solid residue is subsequently removed by filtration to yield a solution of the water extract of camellia sinensis.

(4) The aforementioned skin care product is obtained by mixing the three solutions obtained from Steps 1-3.

### Example 4

A forth example of preparation of the aforementioned skin care product involves the following steps:

(1) In mass ratio, 5 parts of tea and 100 parts of 70% ethanol are added into a reactor and refluxed for 1.5 hours; the solid residue is subsequently removed by filtration to yield a solution of the alcohol extract of camellia sinensis.

(2) The solid residue obtained from Step 1 and 200 parts of vinegar (or 5% acetic acid solution) are added into a reactor and refluxed for 1.5 hours; the solid residue is subsequently removed by filtration to yield a solution of the acid extract of camellia sinensis.

(3) The solid residue obtained from Step 2 and 200 parts of purified water are added into a reactor and refluxed for 1.5 hours; the solid residue is subsequently removed by filtration to yield a solution of the water extract of camellia sinensis.

(4) The aforementioned skin care product is obtained by mixing the three solutions obtained from Steps 1-3.

### Example 5

An example of preparation of a skin care product containing, in mass percentage, the following components: 0.01-5% alcohol extract of camellia sinensis, 0.1-10% acetic acid, 5-40% alcohol, 50-95% water, involves the following steps:

(1) In mass ratio, 10 parts of tea and 100 parts of 70% ethanol is added into a reactor and refluxed for 1.5 hours; the solid residue is subsequently removed by filtration; the filtrate is concentrated, by reduced pressure distillation, to one half of its original volume to yield a concentrated solution of the alcohol extract of camellia sinensis.

(2) The skin care product is obtained upon mixing, in mass ratio, 50 parts purified water, 1 parts of acetic acid, and 15 parts of the concentrated solution of camellia sinensis obtained from Step 1.

### Example 6

An example of preparation of a skin care product containing, in mass percentage, the following components: 0.01-5% water extract of camellia sinensis, 0.1-10% acetic acid, 5-40% alcohol, 50-95% water, involves the following steps:

(1) In mass ratio, 10 parts of tea and 300 parts of purified water is added into a reactor and refluxed for 1.5 hours; the solid residue is subsequently removed by filtration to yield a solution of the water extract of camellia sinensis.

(2) The aforementioned skin care product is obtained upon mixing, in mass ratio, 10 parts ethanol, 2 parts of acetic acid, and 100 parts of the solution obtained from Step 1.

### Example 7

An example of preparation of a skin care product containing, in mass percentage, the following components: 0.01-5% acid extract of camellia sinensis, 0.1-10% acetic acid, 5-40% alcohol, 50-95% water, involves the following steps:

(1) In mass ratio, 10 parts of tea and 100 parts of vinegar (5% acetic acid) is added into a reactor and refluxed for 1.5 hours; the solid residue is subsequently removed by filtration to yield a solution of the acid extract of camellia sinensis.

(2) The aforementioned skin care product is obtained upon mixing, in mass ratio, 100 parts of purified water, 20 parts of ethanol, and 100 parts of the solution obtained from Step 1.

### Example 8

An example of preparation of a skin care product containing, in mass percentage, the following components: 0.01-5% extract of camellia sinensis, 0.01-5% extract of onion, 0.1-10% acetic acid, 5-40% alcohol, 50-95% water, involves the following steps:

(1) In mass ratio, 5 parts of tea and 50 parts of 70% ethanol is mixed and refluxed in a reactor for 1.5 hours; the solid residue is subsequently removed by filtration to yield a solution of the alcohol extract of tea (camellia sinensis).

(2) In mass ratio, 25 parts of onion and 50 parts of 70% ethanol is mixed in a sealed container and sonicated for 30 minutes; the solid residue is subsequently removed by filtration to yield a solution of the alcohol extract of onion.

(3) The skin care product is obtained by mixing the two solutions obtained from Steps 1-2 with 3 parts of acetic acid and 200 parts of purified water.

### Example 9

An example of preparation of a skin care product containing, in mass percentage, the following components: 0.01-5% extract of camellia sinensis, 0.01-5% extract of lemon, 0.1-10% acetic acid, 5-40% alcohol, 50-95% water, involves the following steps:

(1) In mass ratio, 5 parts of tea and 50 parts of 70% ethanol is mixed and refluxed in a reactor for 1.5 hours; the solid residue is subsequently removed by filtration to yield a solution of the alcohol extract of tea (camellia sinensis).

(2) In mass ratio, 25 parts of lemon and 50 parts of 70% ethanol is mixed in a sealed container and sonicated for 30 minutes; the solid residue is subsequently removed by filtration to yield a solution of the alcohol extract of lemon.

(3) The skin care product is obtained upon mixing the two solutions obtained from Steps 1-2 with 1.5 parts of acetic acid and 200 parts of purified water.

### Example 10

An example of preparation of a skin care product containing, in mass percentage, the following components: 0.01-5% extract of camellia sinensis, 0.01-5% extract of onion, 0.01-5% extract of lemon, 0.1-10% acetic acid, 5-40% alcohol, 50-95% water, involves the following steps:

(1) In mass ratio, 5 parts of tea and 50 parts of 70% ethanol is mixed and refluxed in a reactor for 1.5 hours; the solid residue is subsequently removed by filtration to yield a solution of the alcohol extract of tea (camellia sinensis).

(2) In mass ratio, 15 parts of onion and 30 parts of 70% ethanol is mixed in a sealed container and sonicated for 30 minutes; the solid residue is subsequently removed by filtration to yield a solution of the alcohol extract of onion.

(3) In mass ratio, 20 parts lemon and 30 parts of 70% ethanol is mixed in a sealed container and sonicated for 30 minutes; the solid residue is subsequently removed by filtration to yield a solution of the alcohol extract of lemon.

(4) The skin care product is obtained upon mixing the three solutions obtained from Steps 1-3 with 2 parts of acetic acid and 200 parts of purified water.

### Example 11

An example of preparation of a facial mask containing, in mass percentage, the following components: 0.01-5% extract of camellia sinensis, 0.01-5% extract of onion, 0.01-5% extract of lemon, 0.1-10% acetic acid, 5-40% alcohol, 50-95% water, involves the following steps:

(1) In mass ratio, 5 parts of tea and 30 parts of 35% ethanol is mixed and refluxed in a reactor for 1.5 hours; the solid residue is subsequently removed by filtration to yield a solution of the alcohol extract of tea (camellia sinensis).

(2) In mass ratio, 15 parts of onion and 30 parts of 35% ethanol is mixed in a sealed container and sonicated for 30 minutes; the solid residue is subsequently removed by filtration to yield a solution of the alcohol extract of onion.

(3) In mass ratio, 20 parts lemon and 30 parts of 35% ethanol is mixed in a sealed container and sonicated for 30 minutes; the solid residues is subsequently removed by filtration to yield a solution of the alcohol extract of onion.

(4) The three solutions obtained from Steps 1-3 are mixed with 2 parts of acetic acid and 200 parts of purified water.

(5) The solution obtained from Step 4 is applied to the facial mask sheet and packaged to give the final product.

### Example 12

An example of preparation of a skin cream containing, in mass percentage, the following components: 0.01-5% extract of camellia sinensis, 0.01-5% extract of onion, 0.01-5% extract of lemon, 0.1-10% acetic acid, 5-40% alcohol, 50-95% water, involves the following steps:

(1) In mass ratio, 2 parts of lanolin, 0.5 parts of cetyl alcohol, 10 parts of stearic acid, 0.4 parts of potassium hydroxide is mixed in a container and heated to 75° C.

(2) In mass ratio, 80 parts of the skin care product in the aqueous solution form obtained from Example 10 is mixed with 8 parts of propylene glycol in another container and heated to 75° C.

(3) With continuous stirring, the solution of Step 2 is slowly added into the mixture of Step 1; when the addition is completed, the mixture is cooled to room temperature with continuous stirring.

(4) The mixture from Step 3 is passed through a triple roller mill to yield the skin cream product.

It is to be noted that in order to enhance the efficacy of the extraction process, the camellia sinensis is generally ground into smaller particles while onion and lemon are sliced into smaller pieces prior to the extraction process.

What is claimed is:

1. A skin care product contains the following components: 0.01-5% extract of camellia sinensis, 0.1-10% acetic acid, 5-40% alcohol, 50-95% water.

2. A skin care product according to claim 1, which contains 0.01-5% extract of onion.

3. A skin care product according to claim 1, which contains 5-20% onion juice.

4. A skin care product according to claim 1-3, which contains 0.01-5% extract of lemon.

5. A skin care product according to claim 1-3, which contains 5-20% lemon juice.

6. A skin care product according to claims 1-5, which is characterized by: the extract of camellia sinensis is an alcohol extract of camellia sinensis obtained by using an alcohol as the solvent for the extraction process.

7. A skin care product according to claims 1-5, which is characterized by: the extract of camellia sinensis is an acid extract of camellia sinensis obtained by using an acid as the solvent for the extraction process.

8. A skin care product according to claims 1-5, which is characterized by: the extract of camellia sinensis is a water extract of camellia sinensis obtained by using water as the solvent for the extraction process.

9. A skin care product according to claims 1-5, which is characterized by: the extract of camellia sinensis is obtained by using a mixture of alcohol, acid, and water as the solvent for the extraction process.

10. A skin care product according to claims 1-5, which is characterized by: the extract of camellia sinensis is a mixture of the alcohol extract, acid extract, and water extract of camellia sinensis.

11. A skin care product according to claims 1-10, which is in the form of a skin cream.

12. A skin care product according to claims 1-10, which is in the form of a facial mask.

13. A method for producing a skin care product, which includes:
Produce the alcohol extract of camellia sinensis according to the following steps:
i) The camellia sinensis is mixed with an alcohol then heated to a specific temperature for a certain period of time;
ii) The solid residue is removed by filtration to yield a solution of the alcohol extract of camellia sinensis;
iii) The solution may be subjected to reduced pressure distillation to have the solvent removed either completely to yield the pure alcohol extract of camellia sinensis, or partially to yield a more concentrated solution of the alcohol extract of camellia sinensis;
Produce the acid extract of camellia sinensis according to the following steps:
i) The camellia sinensis is mixed with an acid then heated to a specific temperature for a certain period of time;
ii) The solid residue is removed by filtration to yield a solution of the acid extract of camellia sinensis;
iii) The solution may be subjected to reduced pressure distillation to have the solvent removed either completely to yield the pure acid extract of camellia sinensis, or partially to yield a more concentrated solution of the acid extract of camellia sinensis;
Produce the water extract of camellia sinensis according to the following steps:
i) The camellia sinensis is mixed with water then heated to a specific temperature for a certain period of time;
ii) The solid residue is removed by filtration to yield a solution of the water extract of camellia sinensis;
iii) The solution may be subjected to reduced pressure distillation to have the solvent removed either completely to yield the pure water extract of camellia sinensis, or partially to yield a more concentrated solution of the water extract of camellia sinensis;
Mix the alcohol extract, acid extract, and water extract of camellia sinensis with other necessary components such as an alcohol, acetic acid, water, and any optional component according to claims 1-5.

14. A method for producing a skin care product, which involves the following steps:
Produce the extract of camellia sinensis using a mixed solvent consisting of an alcohol, an acid, and water, according to the following steps:
i) The camellia sinensis is mixed with the solvent then heated to a specific temperature for a certain period of time;
ii) The solid residue is removed by filtration to yield a solution of the extract of camellia sinensis;
iii) The solution may be subjected to reduced pressure distillation to have the solvent removed either completely to yield the pure extract of camellia sinensis, or partially to yield a more concentrated solution of the extract of camellia sinensis;
Mix the extract of camellia sinensis with other necessary components, such as an alcohol, acetic acid, water, and any optional component, according to claims 1-5.
